(19) United States
(12) Patent Application Publication
(10) Pub. No.: US 2025/0336505 A1
(43) Pub. Date: Oct. 30, 2025

(54) DATA-DRIVEN WORKPLACE TO IMPROVE HEALTHCARE STAFF RETENTION

(71) Applicant: Insight Direct USA, Inc., Chandler, AZ (US)

(72) Inventor: Michael Griffin, Wayland, MA (US)

(21) Appl. No.: 18/647,579

(22) Filed: Apr. 26, 2024

(51) Int. Cl. G16H 40/20; G06Q 10/105

(52) U.S. Cl. CPC G16H 40/20; G06Q 10/105

(57) ABSTRACT

A method of identifying work conditions likely to cause employee resignation includes receiving a set of attributes for a nurse and receiving a plurality of shift variables. The set of attributes includes one or more attributes that describe the nurse and each shift variable of the plurality of shift variables describes a characteristic of a work condition in a nursing workplace, such that the plurality of shift variables describe a plurality of work conditions. The method further includes predicting a plurality of resignation likelihoods for the plurality of work conditions, identifying at least one work condition of the plurality of work conditions associated with a high likelihood of nurse resignation based on the plurality of resignation likelihoods, and outputting an indication of the at least one work condition.

### FIELD OF THE INVENTION

The present disclosure relates to worker staffing and turnover and, more particularly, for healthcare worker schedules based on worker attributes in order to improve staff retention and reduce worker turnover.

### BACKGROUND

Healthcare providers hire a variety of medical workers to perform a range of tasks, including providing patient care. Medical worker turnover can cause significant disruptions and can require healthcare providers to invest substantial resources to hire replacement workers to ensure that staffing levels are adequate to meet patient needs and other relevant demands. Patient care can also decline while healthcare operations are understaffed.

### SUMMARY

An example of a method of identifying work conditions likely to cause employee resignation includes receiving a set of attributes for a nurse and receiving a plurality of shift variables. The set of attributes includes one or more attributes that describe the nurse and each shift variable of the plurality of shift variables describes a characteristic of a work condition in a nursing workplace, such that the plurality of shift variables describe a plurality of work conditions. The method further includes predicting a plurality of resignation likelihoods for the plurality of work conditions, identifying at least one work condition of the plurality of work conditions associated with a high likelihood of nurse resignation based on the plurality of resignation likelihoods, and outputting an indication of the at least one work condition. The plurality of resignation likelihoods is predicted by simulating, with a simulator, resignation likelihoods for the nurse based on the set of attributes, the plurality of shift variables, and a computer-implemented machine learning model configured to relate resignation likelihood to shift variables and nurse attributes.

An example of a system includes at least one database, a processor, a user interface, and at least one computer-readable memory encoded with instructions. The instructions, when executed, cause the processor to query the at least one database to receive a set of attributes for a nurse attributes including one or more attributes that describe the nurse and to query the at least one database to receive a plurality of shift variables. The set of attributes includes one or more attributes that describe the nurse and each shift variable of the plurality of shift variables describes a characteristic of a work condition in a nursing workplace, such that the plurality of shift variables describe a plurality of work conditions. The instructions, when executed, further cause the processor to predict a resignation likelihood for each of the plurality of work conditions, identify at least one work condition of the plurality of work conditions associated with a high likelihood of nurse resignation based on the predicted resignation likelihoods, and cause the user interface to output an indication of the at least one work condition. The plurality of resignation likelihoods is predicted by simulating, with a simulator, resignation likelihoods for the nurse based on the set of attributes, the plurality of shift variables, and a computer-implemented machine learning model configured to relate resignation likelihood to shift variables and nurse attributes.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a schematic diagram of an example of a system for predictively scheduling healthcare workers.

FIG. 2 is a flow diagram of an example of a method of scheduling healthcare workers suitable for use by the system of FIG. 1

FIG. 3 is a flow diagram of an example of a method of identifying high-risk work conditions suitable for use by the system of FIG. 1

FIG. 4 is a flow diagram of an example of a method of training machine learning algorithms suitable for use with the system of FIG. 1 and the methods of FIGS. 2-3.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

The present disclosure relates to systems and methods for reducing turnover of medical workers. More specifically, the present disclosure relates to systems and methods for creating work schedules for medical workers that are predicted to reduce the likelihood that those medical workers resign. The present disclosure further relates to systems and methods for identifying working conditions that are predicted to significantly or substantially increase the likelihood that medical workers resign (e.g., from employment). The systems and methods described herein can be used to create schedules for any number of medical workers of a healthcare provider. The present disclosure is described generally with respect to nursing workers, but can be adapted to reduce staff turnover for any suitable class of medical worker.

Healthcare providers can be significantly encumbered by high worker turnover. Using nursing workers as an exemplar for explanatory purposes, a healthcare provider can spend considerable financial resources replacing nursing workers lost to resignation. The healthcare provider not only needs to spend resources advertising the position, but also needs to invest further resources and time, including time of both human resources workers and remaining nursing workers, interviewing, selecting replacement nursing hires, training new staff, and onboarding new staff. Although new hires generally have relevant experience and/or training in nursing, significantly resources and time are typically still expended to impart institution- or employer-specific knowledge, practices, guidelines, procedures, etc. to new hires. Further, patient care and experience can significantly decline while new nursing hires are interviewed, hired, trained, and onboarded. Nurse turnover can also cause loss of institutional knowledge, which can further decrease quality of patient care. Due to the aforementioned difficulties, the average time required to replace a nursing worker can exceed three months and can require significant financial investment per worker replaced. For example, hospitals and other healthcare providers in the United States can spend over $50,000 USD on turnover-related costs for a single nursing worker.

In some jurisdictions, nurse turnover can exceed 25% of the workforce each year and healthcare providers can expect 100% nurse turnover approximately every five years. Large healthcare providers can experience especially high volumes of turnover. For example, sufficiently large healthcare providers can experience turnover of more than 2,000 nursing workers each month, which can result in significant monthly costs to the healthcare provider. These costs are typically passed on to patients, significantly increasing patient cost-of-care.

The systems and methods disclosed herein use computer-implemented machine learning models to identify working conditions predicted to improve nursing worker experience and satisfaction in order to reduce nurse turnover. As will be explained in more detail subsequently, the systems and methods disclosed herein are able to predict the impact of individual working conditions on an individual nurse's likelihood of resignation and, further, create work schedules that are predicted to minimize or reduce nurse resignation. The systems and methods disclosed herein use personalized information about each nurse, such as training, education, experience, and/or relevant biographical factors to predict the impact of working conditions on each nurse's likelihood of resignation. Existing techniques of scheduling nurses and other healthcare workers do not attempt to match working conditions to healthcare workers based personalized information about each nurse. The use of personalized information allows the systems and methods disclosed herein to make accurate predictions regarding conditions that nurses are likely to find unsatisfactory or intolerable and, further, to make those accurate prediction without requiring any nursing workers to specifically articulate working conditions as unsatisfactory or intolerable to managers or other supervisory employees. Rather, the systems and methods disclosed herein are able to accurately predict preferences for working conditions even in examples where workers have not been exposed to those working conditions. Advantageously, this allows the systems and methods disclosed herein to be used in a wide variety of healthcare settings to understand likely working condition preferences and create nursing worker schedules according to those preferences, thereby both improving worker experience and reducing turnover-associated costs to healthcare providers.

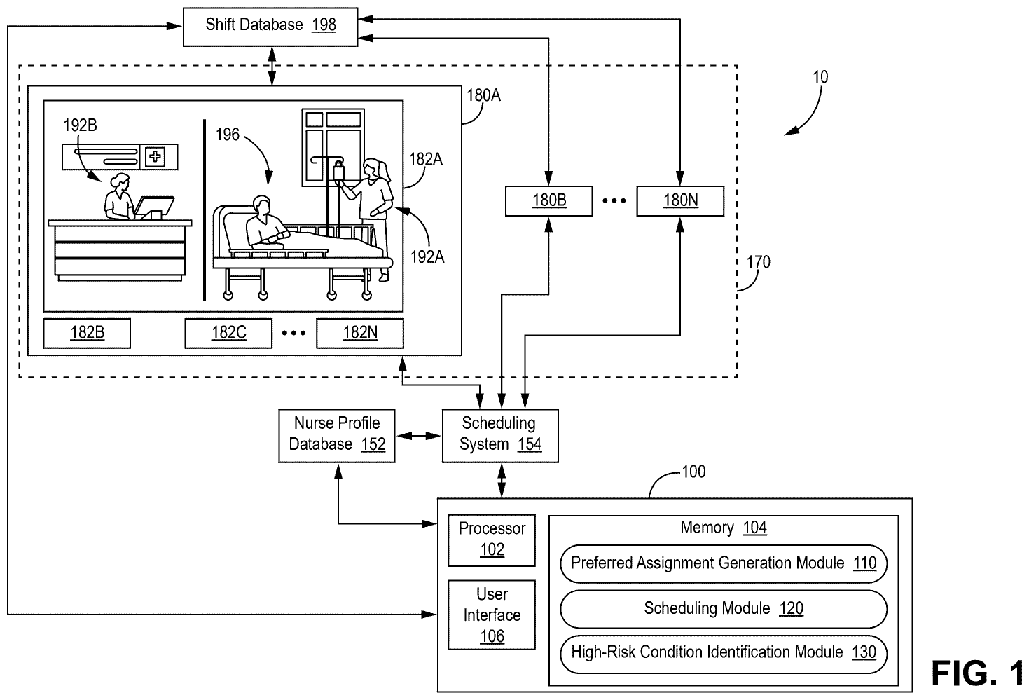

FIG. 1 is a schematic diagram of system 10, which is a system for scheduling nursing staff at one or more healthcare facilities. System 10 includes predictive scheduler 100, which includes processor 102, memory 104, and user interface 106. Memory 104 includes preferred assignment generation module 110 and scheduling module 120. System 10 also includes nurse profile database 152, scheduling system 154, and healthcare system 170. Healthcare system 170 includes hospitals 180A-N, and in the depicted example, healthcare facility 180A includes wards 182A-N. FIG. 1 also depicts nursing employees 192A, 192B as well as patient 196 at ward 182A of healthcare facility 180A.

Predictive scheduler 100 is able to create work schedules for nursing employees that reduce the likelihood of resignation of those nursing employees. Notably, predictive scheduler 100 can create a schedule that reduces the likelihood of resignation of any quantity of nursing employees. Predictive scheduler 100 can create a schedule that, for example, reduces the likelihood of resignation of a single employee, of multiple employees, or of all employees of healthcare system 170. Predictive scheduler 100 includes one or more computer-implemented machine-learning models that are trained to predict a likelihood of nurse resignation based on nurse attributes (e.g., biographical and educational attributes) and working conditions, and can use the computer-implemented machine-learning model(s) to create schedules that are associated with reduces likelihoods of nurse resignation. Predictive scheduler 100 can also, in some examples, use an optimizer or optimization algorithm to create a work schedule that reduces the likelihood of resignation for nurses scheduled therein. As used herein, “nurse attributes” refers to biographical, educational, or other descriptors that describe a nurse, such as a nurse's educational background, physical address, temperament, personality, medical skills, and/or social skills, among other options. Further, as used herein, “working conditions” refers to the conditions that describe a nurse's work assignment, such as the physical location where the work is scheduled to take place (e.g, the healthcare facility 180A-N or facility of a healthcare facility 180A-N), duties to be performed during scheduled work, the hours of scheduled work, patients with whom the nurse is expected to treat or otherwise interact with during scheduled work, the quantity of patients to whom the nurse is assigned during scheduled work, and/or the quantity of other nurses working during scheduled work, among other options. Predictive scheduler 100 can use one or more programs stored to memory 104, such as programs of preferred assignment generation module 110 and scheduling module 120, to perform the functions of predictive scheduler 100 detailed herein.

As used to herein, a “nurse,” “nursing employee,” or “nursing staff member” refers to an employee or contractor of a healthcare facility 180A-N that performs generally medical tasks, such as the treatment of various diseases, patient processing and intake, performing liaison tasks between patients and doctor or physician, providing and coordinating patient care, educating patients and the public about various health conditions, and providing advice and emotional support to patients and their families, as well as assessing, observing, and recording details and symptoms of a patient separate from the performance of those tasks by a doctor or physician. As used herein, a “nurse,” “nursing employee,” or “nursing staff member” does not refer to a doctor, physician, surgeon, or any similar type of medical practitioner. As used herein, “nursing staff” refers to one or more nurses employed or otherwise hired to work at one or more hospitals 180A-N. Predictive scheduler 100 is generally described herein as reducing turnover of nurses of healthcare facilities. However, in other examples, predictive scheduler 100 can be adapted any category of medical worker to reduce turnover and confer the advantages described herein relating to reduced turnover.

Processor 102 can execute software, applications, and/or programs stored on memory 104. Examples of processor 102 can include one or more of a processor, a microprocessor, a controller, a digital signal processor (DSP), an application specific integrated circuit (ASIC), a field-programmable gate array (FPGA), or other equivalent discrete or integrated logic circuitry. Processor 102 can be entirely or partially mounted on one or more circuit boards.

Memory 104 is configured to store information and, in some examples, can be described as a computer-readable storage medium. Memory 104, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, memory 104 is a temporary memory. As used herein, a temporary memory refers to a memory having a primary purpose that is not long-term storage. Memory 104, in some examples, is described as volatile memory. As used herein, a volatile memory refers to a memory that that the memory does not maintain stored contents when power to the memory 104 is turned off. Examples of volatile memories can include random access memories (RAM), dynamic random access memories (DRAM), static random access memories (SRAM), and other forms of volatile memories. In some examples, the memory is used to store program instructions for execution by the processor. The memory, in one example, is used by software or applications running on matching scheduler 100 (e.g., by a computer-implemented machine-learning model or a data processing module) to temporarily store information during program execution.

Memory 104, in some examples, also includes one or more computer-readable storage media. Memory 104 can be configured to store larger amounts of information than volatile memory. Memory 104 can further be configured for long-term storage of information. In some examples, memory 104 includes non-volatile storage elements. Examples of such non-volatile storage elements can include, for example, magnetic hard discs, optical discs, floppy discs, flash memories, or forms of electrically programmable memories (EPROM) or electrically erasable and programmable (EEPROM) memories.

User interface 106 is an input and/or output device and enables an operator to control operation of predictive scheduler 100 and/or other components of system 10. For example, user interface 106 can be configured to receive inputs from an operator and/or provide outputs regarding driver quantity recommendations. User interface 106 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other type of device for facilitating input and/or output of information in a form understandable to users and/or machines.

Predictive scheduler 100 is in in electronic communication with nurse profile database 152 and scheduling system 154, and can access and modify data stored by nurse profile database 152 and scheduling system 154. For example, matching scheduler 100 can modify schedules stored by scheduling system 154 to adjust and update employee schedules to reduce the likelihood of nurse resignation.

Nurse profile database 152 is a database for storing information describing nurses of healthcare facilities 180A-N. Nurse profile database 152 can store any suitable information for describing the nurses of healthcare facilities 180A-N. Nurse profile database 152 can store information in an nurse-by-nurse manner and the data stored for each nurse can be referred to as an “employee profile” or “nurse profile.” Each nurse profile describes one nurse and includes one or more attributes that describe the nurse. For example, a nurse profile can include preferences regarding shift time and shift location (i.e., preferences regarding work at a particular healthcare facility 180A-N or a particular facility at a healthcare facility 180A-N). Additionally and/or alternatively, employee profiles stored by nurse profile database 152 can include information describing employee expertise, training, education, specialties, skill sets, etc. In some examples, the stored nurse profiles can include store biographical information and/or other suitable personal information describing each nurse, such as the nurse's home address and/or descriptors of the nurse's temperament, demeanor, etc. Nurse profile database 152 can be queryable such that processor 102 can query nurse profile database 152 with identifying information for a particular nurse to retrieve the employee profile for that patient. The identifying information can be, for example, a name, employee identification number, and/or government identification number, among other options. Nurse database 152 can be updated nursing staff or other suitable staff of a healthcare facility 180A-N, or another suitable entity, such as a human resources officer of healthcare system 170.

Nurse profile database 152 includes machine-readable data storage capable of retrievably housing stored data, such as database or application data. In some examples, nurse profile database 152 includes long-term non-volatile storage media, such as magnetic hard discs, optical discs, flash memories and other forms of solid-state memory, or forms of electrically programmable memories (EPROM) or electrically erasable and programmable (EEPROM) memories. Nurse profile database 152 can organize data using a relational database management system (RDBMS), object-relational database management system (ORDBMS), columnar database management systems (CDBMS), document-oriented database management systems (DoDBMS) and/or a multi-model database management system (MMDBMS).

Scheduling system 154 creates and manages nurse schedules at healthcare facilities 180A-N of healthcare system 170. Scheduling system 154 is connected to nurse profile database 152 and/or matching scheduler 100 such that scheduling system 154 can electronically communicate with nurse profile database 152 and/or matching scheduler 100, respectively. Scheduling system 154 can be modified by matching schedule 100 and/or can be modified by medical or non-medical staff of a healthcare facility 180A-N, or another suitable entity, such as a human resources officer of healthcare system 170. Scheduling system 154 can store patient appointment information to computer-readable memory substantially similar to memory 104, and further can include processor(s) and/or user interface(s) substantially similar to processor 102 and user interface 106, respectively.

Healthcare system 170 is a business or other organizational entity that includes healthcare facilities 180A-N. Healthcare facilities 180A-N are physical locations where healthcare is provided. Each of healthcare facilities 180A-N corresponds to a discrete, location, or structure that belongs to healthcare system 170. The employees of healthcare system 170 include all employees of healthcare facilities 180A-N, including the nursing staff of healthcare facilities 180A-N. Healthcare system 170 also includes various other employees that do not work specifically for a healthcare facility 180A-N, such as employees in managerial or administrative roles and whose normal duties include the performance of tasks for more than one healthcare facility 180A-N. A healthcare facility 180A-N can be a hospital, clinic, treatment center, or any other suitable type of facility for providing medical advice, diagnosis, prognosis, treatment, etc. System 10 provides patient and employee schedules for all of healthcare facilities 180A-N. In some examples, system 10 can include only one healthcare facility 180A-N and, in yet further examples, system 10 can include fewer or more than the three healthcare facilities 180A-N depicted in in FIG. 1.

Healthcare facility 180A includes wards 182A-N. Each of wards 182A-N corresponds to a different location or sub-location of healthcare facility 180A where different types of patients receive treatment. Each ward 182A-N can correspond to different physical portions or elements of healthcare facility 180A and, in some examples, the different wards 182A-N can share some or all of the physical elements of healthcare facility 180A. Examples of wards 182A-N can include, for example, causality, general ward, special wards, semi-special wards, a critical care unit, an intensive car unit, a surgical intensive car unit, a burn ward, a neonatal intensive care unit, a geriatric ward, and/or a pediatric intensive care unit, among other suitable subdivisions. A nurse of healthcare facility 180A can be assigned to a ward of healthcare facility 180A as the location where the nurse will primarily perform work duties during a scheduled shift. FIG. 1 only depicts the wards of healthcare facility 180A in detail for clarity and explanatory purposes, but each of healthcare facilities 180B-N can also include various wards or other suitable subdivisions. Further, different healthcare facilities of healthcare facilities 180A-N can include different combinations of wards, such that certain nurses of hospital system 180 may be schedules to particular healthcare facilities 180A-N in order to work in particular wards of those healthcare facilities 180A-N.

The depiction of ward 182A in FIG. 1 includes nurses 192A, 192B and patient 196. Nurse 192A and nurse 192B are depicted as performing differing duties within ward 182A. More specifically, nurse 192A is depicted as performing medical duties, such as treating, diagnosing, assessing, observing, or otherwise interacting with patient 196. Nurse 192B is depicted as performing administrative duties. Bars are shown between the scenes depicting nurse 192A and nurse 192B for clarity and explanatory purposes. Further, for clarity and explanatory purposes, only a portion of ward 182A is depicted FIG. 1. It is understood that ward 182A can include additional nurses, patients, rooms, etc. Further, for clarity and explanatory purposes FIG. 1 only depicts ward 182A of healthcare facility 180A in detail for clarity and explanatory purposes, but each of wards 182B-N and/or wards of other healthcare facilities 180B-N can include any number and/or suitable type of patient, nurses, rooms, sub-facilities, etc.

Shift database 198 stores data that describes work conditions and shift requirements for upcoming shifts at each healthcare facility 180A-N of healthcare system 170. Shift database 198 can organize shift information according to any suitable interval of time. For example, shift database 198 can organize shift information to describe shift requirements for different hours, sub-hour time intervals, days, and/or weeks, among other options. Shift database 198 can be queried by predictive scheduler 100 to determine possible work conditions during a shift or during any other suitable time period.

Shift database 198 can store various information describing work and labor requirements during various shifts and/or other suitable time periods. Shift database 198 can store, for example, labor requirements at healthcare system 170 during various time periods, as well as the sublocation of healthcare system 170 (e.g., the healthcare facility 180A-N, building, ward, etc.) where the labor is to be performed. In some examples, shift database 198 can store labor requirements as particular shifts to be performed at healthcare system 170, including the start and stop times of each shift, the sublocation of each shift, and any other suitable information for describing each available shift at healthcare system 170. Shift database 198 can further store information describing the duties available to be performed during each shift and/or time period as well as the sublocation of healthcare system 170 where those duties are available to be performed. Shift database 198 can also store the patients expecting care during each shift and/or time period, including each sublocation of healthcare facility 180A-N of each patient, and/or with particular duties required for the care of each patient, among other options.

As will be explained in more detail subsequently, predictive scheduler 100 can transform shift information retrieved from shift database 198 into shift constraints for a particular shift or another suitable period of time to optimize work conditions for one or more nurses of healthcare system 170. As described previously, it may be desirable for particular quantities of nurses to be present during particular time periods and, further, for subsets of those nurse quantities to perform various duties at various wards, locations, etc. and/or to attend to various patients located at various wards, locations, etc. Predictive scheduler 100 can query shift database 198 to obtain shift requirements for a particular time period, such as the total number of required nurses, the number of nurses required to work at particular sublocations of healthcare system 170 (e.g., individual healthcare facilities 180A-N, wards of healthcare facilities 180A-N, individual buildings or structures of healthcare facilities 180A-N, etc.), the number of nurses required to treat each patient expected during the time period, information describing the patients expecting care during the time period, duties required to be performed during the time period as well as the locations of those duties, or any other suitable information relevant to nurse resignation probability. Predictive scheduler 100 can then transform those shift requirements into various constraints that can constrain a scheduling optimization performed by predictive scheduler 100.

Shift database 198 can be queryable such that predictive scheduler 100 can query shift database 198 with a working condition and receive entries of shift database 198 that include the working condition. For example, shift database 198 can be queried with a specific duties assignment, patient assignment, location assignment, time (e.g., shift start time and/or shift stop time), among other options. As used herein, a “workplace assignment” or “location assignment” refers to a healthcare facility 180A-N and/or a ward of a healthcare facility 180A-N to which a nurse can be scheduled. As used herein, a “duties assignment” refers to an assignment for one or more duties available during a given shift at a given ward and/or healthcare facility 180A-N. As used to herein, a “patient assignment” refers to one or more patients to whom a nurse is assigned during a given shift. A patient assignment can be represented with identifying information for individual patients or can be represented with descriptors that describe those patients. For example, a patient assignment can include temperament or demeanor, relevant health conditions, and/or other information that may be relevant to particular types of tasks that a nurse would perform during a shift. As a particular example, patient assignment information can include age information for patients of a given ward and/or healthcare facility 180A-N, as treatment of different age ranges of patients may involve significantly different skills, tasks, and/or duties performed by a nurse. For example, neonatal care and geriatric care often involve specialized skills that are different than those performed by nurses providing care to a general adult patient population. In some examples, the age information describing a patient population can be stored as duty assignment information in shift database 198.

Shift database 198 can also store information describing all possible working conditions available at healthcare system 170, such as in one or more lists or tables, and can further organize that working condition information into various classes and subclasses, such that shift database 198 can be queried to retrieve all conditions within a particular class or subclass stored to shift database 198. As used herein, a “class” of data refers to a broad category of working conditions, such as location information, shift time information, duties information, and/or patient information, among other options. As used herein, a “subclass” of data refers to a subgrouping of work conditions within a particular class. For example, a subclass of the location class information stored by shift database 198 may be a particular building at a healthcare facility 180A-N or a particular ward at healthcare facility 180A-N. As a further example, a subclass of the patient class information stored by shift database 198 may be a particular category of patient, such as geriatric patients, neonatal patients, obstetrical patients, orthopedic patients, physical therapy patients, etc. As will be explained in more detail subsequently, shift database 198 can be queried to retrieve all possible working conditions of a particular class or subclass in order to identify particular working conditions of that class or subclass that are strongly associated with an increased likelihood of resignation for one or more nurses.

Shift database 198 is shown as a separate component of system 10 in FIG. 1 that is communicatively connected to predictive scheduler 100 and, further, to healthcare facilities 180A-N (via suitable computing devices). In at least some examples, shift database 198 can be substantially integrated with employee scheduling system 154, such that employee scheduling system 154 also maintains the data of shift database 198 and can provide data in response to queries from predictive scheduler 100 or another suitable computing device of system 10.

Preferred assignment generation module 110 includes one or more programs for generating a preferred work assignment for the nurses of healthcare system 170. As used herein, a “work assignment” includes shift conditions (e.g., the times, locations, duties, patients, etc. of each shift) for any suitable number of nurses of healthcare system 170. As will be explained in more detail subsequently, the shift conditions for a nurse of healthcare system 170 can be expressed and/or stored as sets of values for one or more shift variables, where each shift variable represents a work condition, such as a duties assignment, a patient assignment, a location assignment, etc. Preferred assignment generation module 110 can optimize shift variables for any suitable number of nurses of healthcare system 170 using, for example, an optimization algorithm.

Preferred assignment generation module 110 includes at least one computer-implemented machine-learning model configured to predict nurse resignation probability based on nurse attributes (e.g., attributes of nurse profiles stored by nurse profile database 152) and working conditions. Preferred assignment generation module 110 can also include one or more optimization algorithms that can be used to create work assignments that reduce the resignation probability for one or more nurses of healthcare system 170. Each nurse attribute and/or shift variable can be represented as one or more text characters and/or numbers for use as inputs to the computer-implemented machine-learning model. The output of the optimization algorithm(s) of preferred assignment generation module 110 is referred to herein as a “preferred work assignment.” A preferred work assignment includes one or more sets of optimized shift variables that describe upcoming working conditions for one or more nurses. For each nurse, the preferred work assignment can include shift variables that describe a single continuous working period (i.e., a “shift”) and/or for more than one continuous working period for one or more nurses (i.e., multiple “shifts”).

The computer-implemented machine-learning model can be trained using labeled historical nurse turnover data. Healthcare system 170 can use nurse attribute information as well as work schedule information for former and current nursing staff to create training data for training the computer-implemented machine-learning model. More specifically, nurse attribute information can be associated with work schedule information for individual shifts and labeled according to whether the nurse had resigned from healthcare system 170. The labeled data can then be used to train the computer-implemented machine-learning model to make predictions regarding the likelihood of a nurse to resign when the nurse is exposed to particular work conditions at healthcare system 170.

Predictive scheduler 100 can optimize shift variables for each nurse according to limits defined by one or more shift variable constraints. As used herein, a “constraint” or an “optimization constraint” can refer to any suitable constraint, boundary condition, etc. that can be used by an optimization algorithm. The constraints limit the inputs to the computer-machine learning model(s) used by preferred assignment generation module during the optimization. Specifically, the constraints limit inputs describing possible working conditions. Generally, the constraints are configured limit the inputs to the computer-implemented machine-learning model(s) used in workplace assignment optimizations performed by preferred assignment generation module 110 to upcoming, expected, or desired workplace conditions of healthcare system 170. Nurse profile information from nurse profile database 252 defines the nurse attributes that are used as inputs for the computer-implemented machine-learning model(s).

Predictive scheduler 100 can receive the constraints for any number of shift variables from, for example, input at user interface 106. Additionally and/or alternatively, predictive scheduler 100 can receive shift variable constraints by querying other elements of system 10, such as shift database 198. Predictive scheduler 100 can, for example, query shift database 198 to determine work and/or labor requirements for any suitable upcoming time period. The work and/or labor requirements can be, for example, particular time periods during which certain quantities of nurses are desired to be working and/or be on call, duties to be performed during those time periods, patients associated with those duties, patients expecting care during those time periods, locations associated with those duties and/or patients, or any other suitable information for defining the work and labor requirements at healthcare system 170. Predictive scheduler 100 can convert those work and labor requirements into constraints that can be used to constrain optimizations performed by the optimization algorithm. The optimization algorithm can be configured to vary shift variable values according to the boundaries, ranges, etc. defined by the constraints in order to create a preferred work assignment predicted to reduce and/or minimize nurse resignations.

The program(s) of preferred assignment generation module 110 can, for example, optimize shift variables for nurses on an individual basis to reduce individual likelihoods of nurse resignation, and generate a preferred work assignment for each nurse. Additionally and/or alternatively, the program(s) of preferred assignment generation module 110 can optimize working conditions for all nurses. More specifically, the optimization algorithm can be configured to optimize working conditions for a group of nurses to create a set of working conditions that is predicted, according to the computer-implemented machine-learning model, to reduce the average or overall likelihood of resignation for the group. In these examples, the preferred work assignment generated by the program(s) of preferred assignment generation module includes information for the group of nurses.

In examples where preferred assignment generation module is configured to perform a constrained optimization, the optimization algorithm can be configured to vary each shift variable according to the shift constraints to find a combination of working conditions for one or more nurses that reduce or minimize their probability of resignation. For multi-nurse optimizations, the constraints can provide, for example, an upper limit and/or a floor to the number of instances that a particular working condition can occur within a preferred assignment output by the optimization algorithm(s). For example, scheduling all nurses to work day shifts rather than night shifts may result in a lower overall probability of nurse resignation than scheduling some nurses to work night shifts, but would leave night shifts unstaffed at healthcare facilities 180A-N, which is an undesirable result. Accordingly, the optimization algorithm(s) used by preferred assignment generation module 110 can be configured to ensure that a particular number or a range of nurses is staffed during particular time periods. Other examples in which it is useful constrain the number of instances of particular a working condition in a preferred work assignment are possible, and those examples constraints for those working conditions can be created and used in substantially the same manner as described previously.

In at least some examples, the constraints obtained by predictive scheduler 100 describe wok conditions of specific upcoming shifts at healthcare system 170. In these examples, predictive scheduler 100 can receive shift information and can generate constraints based on information for individual shifts. The constraints can be linked such that the optimization performed by predictive scheduler 100 can be used to assign particular nurses to pre-determined shifts at healthcare system 170. For example, predictive scheduler 100 can receive combinations of working conditions, such as patient assignments, duties assignments, workplace assignments, and shift times that correspond to particular shifts available at healthcare system 170. Scheduling system 154 and/or shift database 198 can be configured and/or programmed with information for upcoming shifts and predictive scheduler 100 can query scheduling system 154 and/or shift database 198 to receive upcoming shift information. For example, if a particular kind of work is only available at one healthcare facility 180A-N and if there are particular kinds of patients associated with that work, those conditions can be linked such that the set of working conditions are used as an input to the computer-implemented machine-learning model. The optimization algorithm can be configured to pair sets of working conditions with sets of nurse attributes and to create a combinations of working conditions and nurse attributes that are associated with a reduced or minimized likelihood of nurse resignation for those nurses whose attributes are used in the optimization.

While the shift constraints used by predictive scheduler 100 in constrained optimizations have generally been described herein as specifying working conditions, such as a range of nurse quantities per shift or time period, a range of patient quantities to whom a single nurse can be assigned per shift or in a given time period, a range of available workplace assignments, a range of available duties assignments, a shift length range, and a range of patient assignments, the shift constraints can also define and/or be derived from other scheduling elements. For example, the constraints can also define and/or be derived from a minimum number of hours between scheduled shifts, a maximum number of consecutive overnight shifts, and a maximum number of hours during a given time period. The constraints can be used by an optimization algorithm or another suitable program of preferred assignment generation module 110 to limit the inputs of working conditions accepted by the machine-learning model(s) of preferred assignment generation module 110, such that the outputs of preferred assignment generation module 110 reflect a work assignment for one or more nurses of healthcare system 170 that reduces nurse resignation probability while meeting the needs of the healthcare facilities 180A-N and patients of healthcare system 170.

As described previously, in some examples, an employee or another suitable entity can manually configure predictive scheduler 100 with optimization constraints. In other examples, predictive scheduler 100 can query elements of system 10 to automatically generate and/or retrieve optimization constraints. In some examples, predictive scheduler 100 can query shift database 198 to obtain all constraints for the optimization algorithm of preferred assignment generation module 110. For example, if shift database 198 stores all relevant data for upcoming shifts, predictive scheduler 100 can query shift database 198 to obtain information for generating all optimization constraints. In yet further examples, shift database 198 may only store relevant data about healthcare facilities 180A-N. In these examples, predictive scheduler 100 can query nurse scheduling system 154 to obtain shift time and location information (i.e., facility and/or ward information), and can further query shift database 198 with the shift location information to, for example, obtain duties and patient information for each location.

Predictive scheduler 100 can query nurse profile database 152 to determine nurse attributes for nurses employed by or otherwise contracted or designated to work for healthcare system 170. Predictive scheduler 100 can use the received constraints and nurse attribute information to generate a preferred work assignment for one or more nurses of healthcare system 170.

Scheduling module 120 includes one or more programs for modifying scheduling data stored to nurse scheduling system 154. The program(s) of scheduling module 120 are able to modify data of nurse scheduling system 154 according to the preferred work assignments generated by preferred assignment generation module 110. In some examples, the program(s) of scheduling module 120 can be configured to automatically modify nurse scheduler system 154 after the program(s) of preferred assignment generation module 110 generate a preferred work assignment.

After selecting a preferred employee combination for the patients scheduled during the time period, the program(s) of employee scheduling module 120 can be configured to output the preferred employee combination and/or to modify employee scheduling system 154. The program(s) of employee scheduling module 120 can output the preferred employee combination to allow employees of hospital system 182 to schedule the employees of the employee combination. Additionally and/or alternatively, the program(s) of employee scheduling module 120 can be configured to automatically modify employee scheduling system 154 to schedule the employees of the preferred employee combination. The programs of employee scheduling module 120 can be run iteratively to create employee schedules for as many time periods, shifts, and/or appointment windows as is desirable for a given healthcare facility 180A-N and/or for hospital system 182.

High-risk condition identification module 130 includes one or more programs for identifying high-risk work conditions. As used herein, “high-risk work conditions” refer to those conditions identified by predictive scheduler as being associated with a particularly high likelihood of nurse resignation. The computer-implemented machine-learning model(s) used by preferred assignment generation module 110 can be used to identify or recognize conditions are most likely to result in resignation for individual nurses, or that otherwise are predicted to have the greatest impact on a particular nurse's likelihood of resignation. High-risk condition identification module 130 can include a simulator that is configured to simulate, using the computer-implemented machine-learning model(s) described previously, probabilities of resignation for different combinations of working conditions for individual nurses of healthcare system 170. The simulator can vary conditions for each class or subclass to determine which condition(s), for each class or subclass, contribute most to increasing a nurse's likelihood of resignation. Predictive scheduler 100 can output the working conditions that are identified by the simulator as most predictive of nurse resignation. Predictive scheduler 100 can be configured, for example, to output, for each class and/or subclass, the condition that is predictive to cause the largest increase in a nurse's likelihood of resignation. Additionally and/or alternatively, high-risk condition identification module of predictive scheduler 100 can be configured with a threshold value (e.g., of resignation likelihood) for identifying conditions as high-risk work conditions. Predictive scheduler 100 can output indications of work conditions predicted to result in a likelihood of nurse resignation as, for example, text at user interface 106. Predictive scheduler 100 can identify high-risk work conditions for individual nurses and/or groups of nurses of any suitable size, including a group encompassing all nurses of healthcare system 170 or any subset thereof.

High-risk condition identification module 130 can be configured to query shift database 198 and/or nurse scheduling system 154 to receive a set of possible working conditions available at healthcare system 170 and/or values representing those conditions. High-risk condition identification module 130 can calculate the impact on resignation of all available conditions at healthcare system 170 and can, according to the calculated resignation probabilities, identify conditions that are predicted to result in increased or higher likelihoods of nurse resignation for each nurse of healthcare system 170.

Predictive scheduler 100 can also modify nurse profile data stored to nurse profile database 152 or cause nurse profile database 152 to modify stored nurse profile data to include data describing high-risk work conditions for each nurse. The high-risk work conditions identified for each nurse can be used by nurse scheduling system 154 to schedule nursing workers to various shifts, facilities, etc. Predictive scheduler 100 can use, for example, an optimization algorithm to create a schedule based on the stored nurse identity and high-risk work condition information. The optimization algorithm can be configured to, for example, create a schedule that reduces or minimizes an overall or total number of high-risk work conditions experienced by any suitable group of nurses. The group of nurses can include, for example, all nurses of healthcare system 170 or one or more groups of nurses (e.g., nurses of a particular specialty) that typically have high turnover, among other options. Predictive scheduler 100 can modify employee scheduling system 154 according to the generated schedule using the programs of scheduling module 120.

In some examples, predictive scheduler 100 can, for example, retrieve shift information from shift database 198 and/or employee scheduling system 154 to determine available shifts for nurses of healthcare system 170. The programs of high-risk condition identification module can use high-risk work condition information to create optimized shift assignments that reduce and/or minimize nurse exposure to work conditions identified as high-risk work conditions.

The high-risk work conditions identified by the programs of high-risk condition identification module 130 can also be used to prompt additional follow-up or other interventions to reduce nurse turnover by supervisors, managers, or individuals with similar roles in healthcare system 170. For example, predictive scheduler 100 can create alerts and/or reports, among other options, that indicate shifts where particular employees will be subjected to work conditions that have been identified as high-risk resignation conditions. A supervisor, manager, etc. can view alerts and/or reports generated by predictive scheduler 100 in order to identify employees that should be targeted with additional intervention(s) for reducing resignation likelihood. The intervention(s) can be, for example, one or more conversations to monitor employee happiness and overall satisfaction. In some examples, predictive scheduler 100 may identify work conditions as likely to result in resignation for a particular nurse, but the nurse may nonetheless tolerate the work conditions such that repeated exposure to the work conditions does not result in nurse resignation. In these examples, additional intervention(s) by a supervisor, manager, etc. can be used to evaluate whether a high-risk work condition was accurately identified. If the high-risk work condition is found to have been incorrectly identified, the supervisor, manager, etc. can update the nurse profile information for the nurse (i.e., stored to nurse profile database 152) accordingly.

Advantageously, predictive scheduler 100 is able to create information that can be used to reduce nurse turnover in healthcare system 170. Predictive scheduler 100 can use the optimization algorithm and the constraints to create a work schedule for nurses of healthcare system 170 that reduces or minimizes the likelihood of nurse resignation, advantageously reducing nurse turnover at hospital system 170. Predictive scheduler 100 is also able to use nurse attribute information in order to predictively identify work conditions that are particularly likely to cause nurses of healthcare system 170 to resign. Predictive scheduler 100 can generate preferred work assignments and predictive scheduler 100 can modify and/or create nurse schedules based on the preferred work assignments and/or work conditions identified as likely to result in nurse resignation. In some examples, predictive scheduler 100 can automatically modify and/or create nurse scheduling data, which advantageously allows preferred work assignment and high-risk work condition information to be incorporated into nurse work schedules without additional user input.

As described previously, training and onboarding of newly hired nurses can take significant time and can cause healthcare system 170 to incur significant costs. Further, patient care quality can decline after nurses of healthcare system 170 resign until new hires are trained and onboarded. Accordingly, reducing nurse turnover using schedules created using preferred assignment generation module 110 can advantageously reduce costs associated with hiring replacement workers and, further, can improve patient care quality.

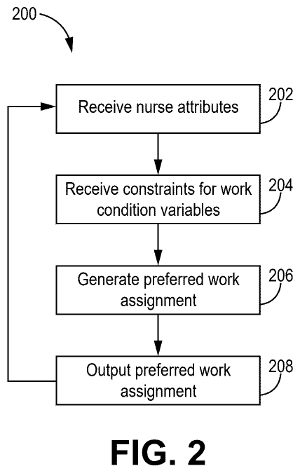

FIG. 2 is a flow diagram of method 200, which is a method of generating a preferred work assignment suitable for use by predictive scheduler 100. Method 200 includes steps 202-208 of receiving nurse attributes (step 202), receiving constraints for shift variables (step 204), generating a preferred work assignment (step 206), and outputting an indication of the preferred work assignment (step 208). Method 200 can be performed by any suitable device, but for clarity, method 200 is generally described herein with respect to system 10 and predictive scheduler 100 (FIG. 1). Method 200 can be performed to generate a preferred work assignment for any number of nurses and, in at least some examples, can be performed to generate a preferred work assignment for all nurses of healthcare system 170. Method 200 is also generally described herein as able to create schedules for nurses of healthcare facilities. However, in other examples, method 200 can be adapted to create work assignment information for any category of medical worker to reduce turnover and confer the advantages described herein relating to reduced turnover.

In step 202, predictive scheduler 100 receives nurse attributes from nurse profile database 152. Predictive scheduler 100 can query nurse profile database 152 to receive nurse profile information for each nurse for which a preferred work assignment is to be created. Predictive scheduler 100 can retrieve nurse attribute information for any suitable number of nurses and, in some examples, can receive nurse attribute information for all nurses of healthcare system 170 and/or one or more healthcare facilities 180A-N, one or more wards of a healthcare facility 180A-N, etc. Predictive scheduler 100 can store the retrieved nurse profile information to memory 104 for use with subsequent steps of method 200. The nurse attribute information can be, for example, the nurse profile information stored to nurse database 152 or any suitable subset of the data stored to nurse database 152.

In step 204, predictive scheduler 100 receives shift constraints for each shift variable to be optimized in step 206. The shift variables describe possible working conditions, such as possible shift start and stop times, possible duties assignments, possible patient assignments, and/or possible workplace assignments, among other options. Predictive scheduler 100 can determine the shift variables to be optimized based on, for example, one or more inputs at user interface 106 and/or one or more programs (e.g., configuration files, etc.) stored to memory 104. Predictive scheduler 100 can then query shift database 198 or another suitable element of system 10 to determine upcoming shift requirements and transform those requirements into constraints suitable for an optimization. In some examples, shift database 198 can be configured to retrievably store shift constraints such that predictive scheduler 100 can receive shift constraints by querying shift database 198 and/or memory 104 of predictive scheduler 100 can be configured to store shift constraints.

The shift constraints can specify, for example, a range of desired nurse quantities in a particular shift or time period. The range can be, in some examples, a particular quantity of nurses. The shift constraints can also specify, for example, a range of workplace assignments, a range of patient quantities to whom a single nurse can be assigned, a range of duties assignments, and a range of patient assignments. The shift constraints can also describe, for example, various legal requirements that can be determined based on the legal jurisdiction(s) in which healthcare system 170 and/or individual healthcare facilities 182A-N are located. For example, the shift constraints can also define a minimum number of hours between consecutive work periods or shifts, a maximum number of overnight shifts, and/or a maximum number of worked hours in a given time period (e.g., a week, a month, etc.), among other options.

In step 206, predictive scheduler 100 generates a preferred work assignment for the nurses for whom nurse attributes were received in step 202. The preferred work assignment generated in step 206 includes shift variables for each nurse that describe working conditions predicted to reduce or minimize the likelihood that those nurses will resign. Predictive scheduler 100 can optimize shift variables for each nurse using an optimization algorithm and the computer-implemented machine-learning model configured to accept nurse attributes and shift variables as inputs and further to output nurse resignation probability (as described previously in the discussion of FIG. 1). The optimization algorithm varies the values of the shift variables for each nurse within the bounds established by the shift constraints and minimizes or reduces nurse resignation probability according to the outputs from the computer-implemented machine-learning model. The optimization algorithm can determine a configuration of shift variables for the nurses (i.e., the nurses for whom attributes were received in step 202) that minimizes or reduces nurse resignation likelihood. The optimization algorithm can be configured to find a local or global minima of nurse resignation likelihood and, further, can be any suitable optimization algorithm. In at least some examples, the optimization algorithm can be a gradient descent algorithm. Additionally and/or alternatively, the optimization algorithm can be an iterative optimization algorithm capable of iteratively optimizing shift variables. The work assignment generated by the optimization can be stored to predictive scheduler 100 for use with subsequent steps of method 200.

In some examples, the optimization algorithm is configured to reduce an overall resignation probability for all nurses subject to the work assignment optimization. The overall resignation probability can be determined as, for example, an average resignation probability for the nurses subject to the optimization in step 206. The optimization can be further constrained, in some examples, to ensure that all individual nurse resignation probabilities are below a particular threshold. Advantageously, this can reduce the likelihood that the optimization algorithm does not produce a work assignment solution that assigns one or more nurses to overly unfavorable work conditions (i.e., work conditions predicted to cause a relatively high likelihood of resignation) in order to reduce the overall likelihood of resignation for all nurses included in the optimization. In yet further examples, the optimization algorithm can be configured to find a solution that only reduces the resignation probability of each nurse subject to the optimization below a particular threshold rather than a solution that reduces or minimizes an overall or average likelihood of resignation.

The preferred work assignment produced by the optimization algorithm includes at least one set of one or more optimized shift variables for each nurse for whom nurse attributes was received in step 202 (i.e., for each nurse of the nurses for whom shift variables were optimized). Each set of shift variables describes the working conditions for a single continuous work period or shift and corresponds to a single nurse. Each set of shift variables can include shift variable information describing, for example, a workplace assignment, a duties assignment, shift start and stop times, and/or a patient assignment, among other options.

As shift variables correspond to different work conditions, the shift variables of the preferred work assignment can be used to create an employee schedule that is predicted to result in a lower rate of nurse turnover than existing methods of scheduling nursing staff. In at least some examples, the shift variables of the preferred work assignment can be used to create an employee schedule predicted to result in a minimum rate of nurse turnover.

The optimization performed in step 206 can be for any given period of time as defined by the shift constraints received in step 204. In at least some examples, the work assignment produced in step 206 can describe work conditions (i.e., as shift variables) for multiple shifts having non-identical start and/or stop times. Two or more shifts of the preferred work assignment shift can be partially overlapping, such that part of each shift includes the same time range and, further, such that a remaining and non-overlapping part of each shift does not include the same time range. Additionally and/or alternatively, two or more shifts of the preferred work assignment can be fully-overlapping, such that the time ranges of the shifts are the same. Additionally and/or alternatively, two or more shifts of the preferred work assignment can be non-overlapping, such that no time ranges of the shifts are the same. In at least some examples, the preferred work assignment produced in step 206 can cover a sufficiently large time range that the preferred work assignment includes multiple consecutive shifts for one or more nurses. The minimum time between consecutive shifts for each nurse can be defined by, for example, one or more shift constraints of the shift constraints received in step 204.

In step 208, predictive scheduler 100 outputs an indication of the preferred work assignment. The indication can include, for example, numeric data representative of the sets of optimized shift variables. Additionally and/or alternatively, the indication can include one or more alphanumeric characters that represent the sets of optimized shift variables and/or the work conditions corresponding to the optimized shift variables. Predictive scheduler 100 can cross-reference one or more tables, arrays, databases, etc. in order to transform numeric shift variable data into human-readable alphanumeric text data that describes the work conditions represented by the optimized shift variables. In at least some examples, the indication output in step 208 is text data.

The indication can be output to, for example, user interface 106 of predictive scheduler 106. A user (e.g., an employee of healthcare system 170) can use the work assignment information output at user interface 106 to create a schedule according to the optimized shift variables of the work assignment. Additionally and/or alternatively, predictive scheduler 100 can output the indication of the preferred work assignment by modifying work schedule data stored to scheduling system 154 and/or by causing scheduling system 154 to modify stored work schedule data according to the shift variable information of the preferred work assignment. More specifically, the work schedule data stored to scheduling system 154 can be modified according to the workplace assignments, duties assignments, shift start and stop times, patient assignments, etc. described by the optimized shift variables of the preferred work assignment. In at least some examples, predictive scheduler 100 can be configured to automatically modify and/or cause scheduling system 154 to modify work schedule data according to the preferred work assignment generated in step 206.

Advantageously, method 200 allows for the construction of nurse work schedules, including assignments for specific duties, patients, locations (e.g., healthcare facilities 182A-N, wards of healthcare facilities 182A-N, etc.), and/or other suitable work conditions, that are predicted to reduce and/or minimize nurse turnover of nurses of healthcare system 170. Further, the schedules created using method 200 are constrained (i.e., via constraints received in step 204) such that the work requirements of the healthcare facilities 182A-N of healthcare system 170 are met. That is, the schedules created using method 200 can reduce and/or minimize nurse turnover while also ensuring that patients of healthcare facilities 182A-N have care needs met, that all necessary duties at healthcare facilities 182A-N are performed, and that healthcare facilities 182A-N are adequately staffed at various times of day based on expected patient demand and/or needs. Method 200 also can advantageously reduce costs associated with recruiting and training new nursing staff. Recruitment, training, and onboarding of nursing staff can require significant financial investment. By reducing overall nurse turnover, the schedules created using method 200 can also reduce the likelihood that a new nurse will need to be recruited, trained, and otherwise onboarded to healthcare system 170 to replace a nurse who has resigned. Further, patients can experience significant disruptions to care as new nursing staff are trained and onboarded. Accordingly, the reductions to nurse turnover provided by method 200 can also provide improvements to patient care. Method 200 can be repeated any suitable number of times in order to schedule workers over any suitable time frame. For example, each iteration of method 200 can schedule workers for a given day or a given week, and method 200 can be performed multiple times in order to create nurse work schedules for multiple days or multiple weeks, respectively.

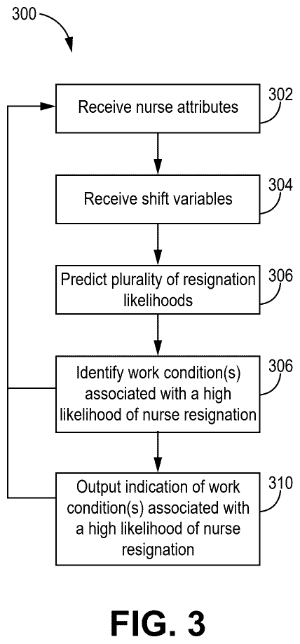

FIG. 3 is a flow diagram of method 300, which is a method of identifying high-risk work conditions suitable for use by predictive scheduler 100. Method 300 includes steps 302-310 of receiving nurse attributes (step 302), receiving shift variables (step 304), predicting a plurality of resignation likelihoods (step 306), identifying one or more work conditions associated with a high likelihood of nurse resignation (step 308), and outputting an indication of the work condition(s) associated with a high likelihood of nurse resignation (step 310). Method 300 can be performed by predictive scheduler 100 and, more specifically, can be performed by the programs of module high-risk condition identification module 130. Method 300 is generally described herein with respect to nurses of healthcare facilities. However, in other examples, method 300 can be adapted to predictively identify high-risk work conditions for any category of medical worker to reduce turnover and confer the advantages described herein relating to reduced turnover.

In step 302, predictive scheduler 100 receives nurse attributes. Step 302 can be performed in substantially the same way as step 202 described previously and with respect to method 200.

In step 304, predictive scheduler 100 receives shift variables describing potential working conditions for the nurse at hospital system 170. The shift variables can describe various workplace assignments, duties assignments, shift start and stop times, and/or patient assignments, among other options. Predictive scheduler 100 can receive shift variables via, for example, one or more user inputs at user interface 106. Additionally and/or alternatively, predictive scheduler 100 can query shift database 198 or another suitable element of system 10 to obtain shift variables describing possible work conditions for upcoming shifts.

In step 306, predictive scheduler 100 predicts a plurality of resignation likelihoods for the nurse for whom nurse attributes were received in step 302. Predictive scheduler 100 can use a computer-implemented machine-learning model configured to accept nurse attributes and one or more shift variables as inputs and to output predicted likelihoods of resignation to generate the plurality of resignation likelihoods in step 306. The computer-implemented machine-learning model can be the same model used for method 200 (FIG. 2) or can be a different computer-implemented machine-learning model. In all examples, each predicted likelihood of resignation describes the amount that a single work condition contributes to the likelihood of resignation for a nurse and, accordingly, can be used to identify work conditions that have a disproportionate and/or high impact on a nurse's likelihood of resignation.

The computer-implemented machine-learning model can be configured to accept a single shift variable as an input or can be configured to accept multiple shift variables as inputs. In examples where the computer implemented machine learning model is configured to accept a single shift variable as an input, the resignation likelihood output by the computer-implemented machine-learning model describes the relative amount that the work condition represented by the shift variable contributes to the nurse's likelihood of resignation. In examples where more the computer-implemented machine-learning model is configured to use more than one shift variable as an input, each shift variable belonging to a different class or subclass of shift variables as defined by the data stored to shift database 198. In these examples, predictive scheduler 100 can be configured to vary a single shift variable while using a baseline or constant set of values for the other shift variables, thereby allowing the predicted likelihood of resignation created in step 306 to each describe the relative amount that a single work condition (i.e., a single shift variable representing the work condition) contributes to a nurse's likelihood of resignation.

In step 308, predictive scheduler 100 identifies one or more work conditions associated with a high likelihood of nurse resignation. Predictive scheduler 100 identifies high-risk work conditions based on the resignation likelihoods predicted in step 306. Predictive scheduler 100 can, for example, use a threshold value to determine high likelihood resignation conditions. For example, predictive scheduler 100 can classify all work conditions having a likelihood of resignation above a particular value as high-risk work conditions. As a further example, predictive scheduler 100 can select the work condition(s) having the highest likelihood of nurse resignation as the high-risk work conditions. Predictive scheduler 100 can be configured to identify a particular number of work conditions predicted to have the greatest contribution toward nurse resignation in step 306. Further, predictive scheduler 100 can be configured to identify a particular number of work conditions out of all work conditions analyzed in step 306 and/or to identify a particular number of work conditions per class or subclass of work conditions.

In step 310, predictive scheduler 100 outputs an indication of the high-risk work condition(s) identified in step 308. The indication can include numeric data representative of the shift variable value(s) representative of the high-risk work condition(s) and/or alphanumeric characters that represent the shift variables value(s) and/or the high-risk work condition(s). Predictive scheduler 100 can cross-reference one or more tables, arrays, databases, etc. in order to transform numeric shift variable data into human-readable alphanumeric text data that describes the work conditions represented by the optimized shift variables. In at least some examples, the indication output in step 310 is text data.

The indication can be output to, for example, user interface 106 of predictive scheduler 106. A user (e.g., an employee of healthcare system 170) can use the high-risk work condition information output in step 310 to create a schedule that does not place nurses in conditions that expose those nurses to work conditions predicted identified in step 308 (i.e., conditions significantly likely to increase the likelihood those nurses resign from healthcare system 170). The indication can also be used by a manager, human resources employee, supervisor, and/or similar individual in a supervisory role as a signal to determine whether an existing schedule exposes any nurses to high-risk work conditions. If so, the supervisory employee can provide additional support, follow-up, etc. to those nurses in order to mitigate any increase to resignation likelihood caused by exposure to a work condition identified in step 308.

In at least some examples, the indication output in step 310 can be a schedule that reduces or minimizes the exposure of nurses to conditions identified in step 308. Steps 302-308 can be repeated any suitable number of times to identify high-risk work conditions for any suitable population of nurses. Predictive scheduler 100 can then use an optimization algorithm to create a schedule (e.g., a work assignment for nurses of healthcare system 170) that reduces or minimizes the likelihood that the scheduled nurses are exposed to conditions identified in step 308. Predictive scheduler 100 can then output the schedule in step 310 by, for example, modifying work schedule data stored to scheduling system 154. Additionally and/or alternatively, predictive scheduler 100 can output the schedule in step 310 to user interface 106 as text or in another suitable form (e.g., one or more icons, etc.). In at least some examples, predictive scheduler 100 can be configured to automatically modify and/or cause scheduling system 154 to modify work schedule data according to work conditions identified in step 308.

Steps 302-308 and/or 302-310 can be repeated for any suitable number of nurses working at healthcare system 170 to identify high-risk work conditions for those nurses. Based on operational need or user preference, separate indications can be output for each nurse (i.e., with each indication including the high-risk work conditions for a single nurse) or a single indication can be output that includes all high-risk work conditions for all nurses analyzed using steps 302-308 of method 300.

Advantageously, method 300 allows for the identification of work conditions that are particularly likely to increase a nurse's likelihood of resignation. Individuals and supervisory roles can use information obtained by method 300 to stage additional interventions to mitigate any impact from exposure to conditions predicted particularly increase resignation likelihood. Further, the information generated using method 300 can be used to generate schedules. Accordingly, the information created using method 300 can be used to reduce and/or minimize nurse turnover. As explained previously, reducing overall nurse turnover also reduces the likelihood that a new nurse will need to be recruited, trained, and otherwise onboarded to healthcare system 170 to replace a nurse who has resigned. Accordingly, method 300 reduces costs associated with hiring and onboarding new staff. Further, as patients can experience significant disruptions to care as new nursing staff is trained and onboarded, the reductions to nurse turnover provided by method 300 can advantageously reduce the incidence that training and onboarding of new nurses affects patient care.

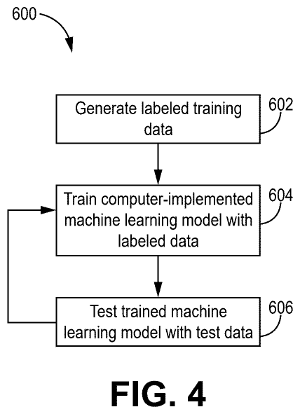

FIG. 4 is a flow diagram of method 600, which is a method of training a computer-implemented machine-learning model suitable for use with method 200 (FIG. 2) and method 300 (FIG. 3) as well as by predictive scheduler 100 (FIG. 1). Method 600 includes steps of generating training data (step 602), training the computer-implemented machine learning model with the training data (step 604), and testing the trained computer-implemented machine learning model with test data (step 606). Method 600 is a method of supervised learning that can be used to train any suitable computer-implemented machine learning model for use with any of methods 200, 300.

In step 602, the training data is generated. For training the computer-implemented machine learning model(s) used in method 200, 300, training data includes nurse profile and/or attribute information for any number of former and current nurses of healthcare system 170 as well as data describing work conditions of shifts that those nurses have worked. Work condition and nurse profile/attribute information can be labeled according to whether the nurse resigned. Nurse resignation can be as a value varying between 0 and 1, among other options. The training data can be generated by analyzing and compiling historical nurse employment data and extracting nurse attribute information, working condition information, and whether the nurse resigned from healthcare system 170.

In step 604, the labeled data is used to train the computer-implemented machine learning model to predict numbers of missed bags based on driver quantities and flight parameters. As used herein, “training” a computer-implemented machine learning model refers to any process by which parameters, hyper parameters, weights, and/or any other value related model accuracy are adjusted to improve the fit of the computer-implemented machine learning model to the training data. The labeled data can be transformed by, for example, one or more programs and/or one or more other trained machine learning models before it is used for training in step 604.

In step 606, the trained computer-implemented machine learning model is tested with test data. The test data used in step 606 does not include labeled patient outcome scores, but otherwise is substantially the same type of data as used in step 602. Accordingly, the test data is unlabeled data that can be used to qualify and/or quantify performance of the trained computer-implemented machine learning model. More specifically, a human or machine operator can evaluate the performance of the machine learning model by evaluating the fit of the model to the test data. Step 606 can be used to determine, for example, whether the machine learning model was overfit to the labeled data during model training in step 604.

As depicted in FIG. 4, steps 604 and 606 can be performed iteratively to improve the performance of the machine learning model. More specifically, if the fit of the model to the unlabeled data determined in step 606 is undesirable, step 606 can be repeated to further adjust the parameters, hyper parameters, weights, etc. of the model to improve the fit of the model to the test data. Step 606 can then be repeated with a new set of unlabeled test data to determine how the adjusted model fits the new set of unlabeled test data. If the fit continues to be undesirable, further iterations of steps 604 and 606 can be performed until the fit of the model becomes desirable.

Method 600 can advantageously be used to train any machine learning model described herein. More generally, the systems and methods disclosed herein advantageously allow for the training and use of machine learning models that can be used to predict healthcare worker resignation likelihoods. The systems and methods disclosed herein can be used to schedule healthcare workers and to identify high-risk work conditions. As described previously, the systems and methods disclosed herein can be used to reduce healthcare worker turnover, which can reduce healthcare costs both to providers and patients as well as improve quality of patient care.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of identifying work conditions likely to cause employee resignation, the method comprising:
receiving a set of attributes for a nurse, the set of attributes including one or more attributes that describe the nurse;
receiving a plurality of shift variables, each shift variable of the plurality of shift variables describing a characteristic of a work condition in a nursing workplace, such that the plurality of shift variables describe a plurality of work conditions;
predicting a plurality of resignation likelihoods for the plurality of work conditions by simulating, by a simulator, resignation likelihoods for the nurse based on the set of attributes, the plurality of shift variables, and a computer-implemented machine learning model configured to relate resignation likelihood to shift variables and nurse attributes;
identifying at least one work condition of the plurality of work conditions associated with a high likelihood of nurse resignation based on the plurality of resignation likelihoods; and
outputting an indication of the at least one work condition.

2. The method of claim 1, wherein:
the plurality of shift variables belong to a plurality of variable classes; and
identifying the at least one work condition of the plurality of work conditions comprises identifying at least one work condition belonging to each variable class of the plurality of variable classes.

3. The method of claim 2, wherein the plurality of variable classes includes at least one of a duties class, a work location class, a shift time class, and a patient class.

4. The method of claim 3, wherein:
each variable class of the plurality of variable classes includes at least one variable subclass, such that the plurality of variable classes comprises a plurality of variable subclasses; and
identifying the at least one work condition of the plurality of work conditions comprises identifying at least one work condition belonging to each variable subclass of the plurality of variable subclasses.

5. The method of claim 1, wherein outputting the indication of the at least one work condition comprises scheduling the nurse to a shift that does not include the at least one work condition.

6. The method of claim 5, wherein scheduling the nurse comprises modifying electronic data of an electronic scheduling system.

7. The method of claim 6, wherein the set of attributes includes at least one of education, experience, age, gender, and marital status.

8. The method of claim 1, and further comprising training the computer-implemented machine learning method with training data, the training data comprising historical job retention data for a plurality of nurses and nurse attributes for the plurality of nurses.

9. The method of claim 1, wherein identifying the at least one work condition comprises comparing the plurality of resignation likelihoods to a threshold resignation likelihood.

10. The method of claim 1, wherein identifying the at least one work condition comprises identifying a work condition of the plurality of work conditions having the greatest resignation likelihood the plurality of resignation likelihoods.

11. A method comprising:
receiving a first set of attributes for a first nurse, the first set of attributes including one or more attributes that describe the first nurse;
receiving a second set of attributes for a second nurse, the second set of attributes including one or more attributes that describe the second nurse;
receiving a plurality of shift variables, each shift variable of the plurality of shift variables describing a characteristic of a work condition in a nursing workplace, such that the plurality of shift variables describe a plurality of work conditions;
generating a first plurality of resignation likelihoods for the first nurse by simulating, by a simulator, resignation likelihoods based on the first set of attributes, the plurality of shift variables, and a computer-implemented machine learning model configured to relate resignation likelihood to shift variables and nurse attributes, wherein each resignation likelihood of the first plurality of resignation likelihoods corresponds to a work condition of the plurality of work conditions;
generating a second plurality of resignation likelihoods for the second nurse by simulating, by a simulator, resignation likelihoods based on the second set of attributes, the plurality of shift variables, and a computer-implemented machine learning model configured to relate resignation likelihood to shift variables and nurse attributes, wherein each resignation likelihood of the second plurality of resignation likelihoods corresponds to a work condition of the plurality of work conditions;
identifying, based on the first plurality of resignation likelihoods, a first work condition of the first plurality of work conditions associated with a high likelihood of resignation for the first nurse;
identifying, based on the second plurality of resignation likelihoods, a second work condition of the second plurality of work conditions associated with a high likelihood of resignation for the second nurse; and
outputting an indication of the first work condition and the second work condition.

12. The method of claim 11, wherein outputting the indication of the first work condition and the second work condition comprises scheduling the first nurse based on the first work condition and scheduling the second nurse based on the second work condition.

13. The method of claim 12, wherein:
scheduling the first nurse based on the first work condition comprises scheduling the first nurse to a first shift that does not include the first work condition; and
scheduling the second nurse based on the second work condition comprises scheduling the nurse to a shift that does not include the second work condition.

14. The method of claim 12, where scheduling the first nurse and scheduling the second nurse together comprises generating a preferred nurse schedule by using an optimization algorithm, the optimization algorithm configured to create a schedule that includes a first shift for the first nurse that does not include the first work condition and a second shift for the second nurse that does not include the second work condition.

15. A system comprising:
at least one database;
a processor;
a user interface; and
at least one computer-readable memory encoded with instructions that, when executed, cause the processor to:
query the at least one database to receive a set of attributes for a nurse, the set of attributes including one or more attributes that describe the nurse;
query the at least one database to receive a plurality of shift variables, each shift variable of the plurality of shift variables describing a characteristic of a work condition in a nursing workplace, such that the plurality of shift variables describe a plurality of work conditions;
predict a resignation likelihood for each of the plurality of work conditions by simulating, by a simulator, the resignation likelihood for each of the plurality of work conditions for the nurse based on the set of attributes, the plurality of shift variables, and a computer-implemented machine learning model configured to relate resignation likelihood to shift variables and nurse attributes;
identify at least one work condition of the plurality of work conditions associated with a high likelihood of nurse resignation based on the predicted resignation likelihoods; and
cause the user interface to output an indication of the at least one work condition.

16. The system of claim 15, wherein:
the plurality of shift variables belong to a plurality of variable classes; and
the instructions, when executed, cause the processor to identify the at least one work condition of the plurality of work conditions by identifying at least one work condition belonging to each variable class of the plurality of variable classes.

17. The system of claim 16, wherein the plurality of variable classes includes at least one of a duties class, a work location class, a shift time class, and a patient class.

18. The system of claim 17, wherein the system further comprises an electronic scheduling system and the instructions, when executed, further cause the processor to modify electronic data of the electronic storage system to schedule the first nurse to a shift that does not include the at least one work condition.

19. The system of claim 18, wherein the set of attributes includes at least one of education, experience, age, gender, and marital status.

20. The system of claim 18, wherein in instructions, when executed, cause the processor to:
retrieve a threshold resignation likelihood from the at least one computer-readable memory; and
identify the at least one work condition by comparing the predicted resignation likelihoods to the threshold resignation likelihood.
